(19) United States
(12) Patent Application Publication
(10) Pub. No.: US 2025/0170068 A1
(43) Pub. Date: May 29, 2025

(54) Encapsulation Method Using Extracellular Vesicle as Carrier

(71) Applicant: National Cheng Kung University, Tainan City (TW)

(72) Inventors: Wei-Hsuan Hsu, Tainan City (TW); Qiao-Yu Hsu, New Taipei City (TW); Bao-Hong Lee, Tainan City (TW)

(21) Appl. No.: 18/789,139

(22) Filed: Jul. 30, 2024

(30) Foreign Application Priority Data
Nov. 29, 2023 (TW) 112146342

(51) Int. Cl. A61K 9/48; A61K 38/44

(52) U.S. Cl. CPC A61K 9/4833; A61K 9/4816; A61K 38/44

(57) ABSTRACT

A novel encapsulation method using extracellular vesicles as carriers. When encapsulating required substances, high pressure processing (HPP) is used to greatly increase the encapsulating efficiency of extracellular vesicles, so as to improve the low encapsulating efficiency of traditional passive encapsulation methods. The method is able to improving the use efficiency of required substances, and also having the advantage of the capability to be mass-produced.

### CROSS-REFERENCE TO RELATED APPLICATION

This application claims the benefit of Taiwan Patent Application No. 112146342, filed on Nov. 29, 2023, in the Taiwan Intellectual Property Office, the disclosure of which is incorporated herein in its entirety by reference.

### BACKGROUND OF THE INVENTION

### 1. Field of the Invention

The present invention generally relates to a novel encapsulation method using extracellular vesicles as carriers, in particular to a novel encapsulation method using extracellular vesicles as carriers which utilizes high pressure processing (HPP).

### 2. Description of the Related Art

Extracellular vesicles (EV) are membranous vesicles obtained from cells, representing endogenous transmission mechanisms between cells.

In general, a cell (i.e. a donor cell) may release various forms of extracellular vesicles, including exosomes (with a diameter ranging from 50 to 150 nm) and microvesicles (with a diameter ranging from 50 to 500 nm). On the other hand, a receptor cell will absorb the released extracellular vesicles through endocytosis, membrane fusion, or specific ligand-receptor internalization, so as to transmit the inclusions (e.g. nucleic acids, lipopolysaccharides, proteins and/or lipids) of the extracellular vesicles to the receptor cell.

Based on its high biocompatibility, low immunity, nanometer size and penetrability of major biological barriers (such as blood-brain barrier), extracellular vesicles can also be used as a candidate therapy for drug delivery. However, its therapeutic efficacy often varies with factors such as the type of disease, severity, and extracellular vesicles characteristics (such as the cell origin, size, and content of the extracellular vesicles). Therefore, in order to improve and increase its therapeutic applicability, pre-conditional culture stimulation or post-isolation incorporation methods have been utilized to coat or encapsulate small molecules, peptides, nucleic acids, enzymes, aptamer and/or scaffold.

Therefore, using extracellular vesicles as an encapsulation carrier has many advantages such as provide good bioavailability and biocompatibility, and can be used for drug entrapment. In addition, surface modification of the extracellular vesicles can provide the ability to cross biological barriers, and provide targeting ability to avoid being captured by immune cells and evade degradation by the immune system.

However, extracellular vesicles still have shortcomings remained to be improved. For example, safety concerns may arise when using extracellular vesicles from cancer cells. In addition, extracellular vesicles of cells are not easy to culture and need to consider issues such as cell density, subculture, and harvesting frequency. Furthermore, extracellular vesicles from stem cells are expensive and suffer low mass production rate.

On the other hand, the current methods of encapsulating drugs in extracellular vesicles are mainly divided into two types, such as passive encapsulation and active encapsulation; passive encapsulation is to culture the drug with host cells or extracellular vesicles, the encapsulation process may be simple but the drug encapsulating efficiency is low; active encapsulation uses ultrasound or extrusion to deform the extracellular vesicles for the drug to enter, although the encapsulating efficiency using ultrasound is the highest among these methods, but the yield is low.

In summary, the current extracellular vesicles encapsulation process and its encapsulating efficiency cannot achieve commercialization requirements of encapsulating large amount of drugs with high yields. Therefore, it is necessary to find a better encapsulation method to improve the poor drug encapsulating efficiency and low yield, so as to improve the efficiency of drug use and enable mass production.

### SUMMARY OF THE INVENTION

Therefore, it is a primary objective of the present invention to improve the method of using extracellular vesicles as carriers to encapsulate the required drugs, so as to improve the encapsulating efficiency of the drugs.

High Pressure Processing (HPP), also known as High hydrostatic pressure processing or Ultra high pressure processing, is characterized in that the volume of the object to be treated will be compressed during the high-pressure treatment process. The extremely high static pressure generated by ultra-high pressure will not only affect the shape of the cell, but also cause the non-covalent bonds such as hydrogen bonds, ionic bonds, and hydrophobic bonds forming the three-dimensional structure of biomacromolecules to change, for example, causing protein coagulation, starch denaturation, or enzyme inactivation or activation.

Therefore, the inventor(s) of the present invention attempts to introduce high pressure processing (HPP) into the encapsulation process of extracellular vesicles, using water as the medium, and pressing the required substance (substance to be encapsulated) with an ultra-high pressure of 0˜600 mPa into the extracellular vesicles to confirm whether high pressure processing can improve encapsulating efficiency and yield without destroying the structure of the extracellular vesicles.

To achieve the foregoing objective, the present invention provides an improved novel encapsulation method using extracellular vesicles as carriers. The novel encapsulation method includes the following steps:
Step S1: isolating the extracellular vesicles;
Step S2: mixing the isolated extracellular vesicles with a required substance to form a first mixture; and
Step S3: processing the first mixture using high pressure processing to obtain the extracellular vesicles encapsulating the required substance.

In a preferred embodiment of the present invention, the extracellular vesicles may be obtained from cells or microorganisms. Preferably, the source of the extracellular vesicles can be Lactobacillus or stem cells (SC); more preferably, the Lactobacillus is Lactobacillus gasseri (L. gasseri), and the stem cells are mesenchymal stem cells (MSC), but are not limited thereto.

In a preferred embodiment of the present invention, the required substance may be selected from small molecules, peptides, nucleic acids, enzymes, aptamers and/or scaffolds, but are not limited thereto. The feature of the novel encapsulation method requested in this application is to introduce high pressure processing to improve the encapsulating efficiency and yield of extracellular vesicles so that they can be mass-produced. This is an improvement in the manufacturing process and thus is not directly related to extracellular vesicles or the required substances to be encapsulated.

To achieve the foregoing objective, the present invention further provides an electronic device comprises a main body, a processing unit and a movable touch device, wherein the movable touch device comprises a casing, a first conductive layer, a second conductive layer and a communication interface unit. The casing has an external surface and an internal surface opposite to the external surface. The first conductive layer is deposed on the external surface of the casing, and a first touch sensor circuit is formed on the external surface of the casing. The second conductive layer is disposed on the internal surface of the casing, and a second touch sensor circuit is formed on the internal surface of the casing. The communication interface unit is installed on the casing for electrically coupling the first conductive layer and the second conductive layer to the electronic device.

The technical features of the invention will be described in detail below with specific embodiments and accompanying drawings, so that those with ordinary knowledge in the art can easily understand the purpose, technical features and advantages of the invention.

### BRIEF DESCRIPTION OF THE DRAWINGS

The exemplary embodiments of the present invention will now be described in more details hereinafter with reference to the accompanying drawings that show various embodiments of the invention as follows for better understanding. However, these embodiments should not be construed as limiting the invention to specific embodiments, but are merely for illustration and explanation.

FIG. 1 is a flow chart of the encapsulation method using extracellular vesicles as carriers according to the present invention;

FIG. 2 is a schematic diagram of the encapsulating efficiency of extracellular vesicles encapsulating the required substance using Lactobacillus as the source of extracellular vesicles and obtained through passive encapsulation and under different pressures; wherein LGEV represents extracellular vesicles of Lactobacillus, CEV+P represents extracellular vesicles encapsulating catalase through passive encapsulation, CEV+0 mPa represents extracellular vesicles encapsulating catalase under 0 mPa pressure, and CEV+300 mPa represents extracellular vesicles encapsulating catalase under 300 mPa pressure;

FIG. 3 is a schematic diagram of the encapsulating efficiency of extracellular vesicles encapsulating the required substance using mesenchymal stem cells as the source of extracellular vesicles and obtained through passive encapsulation and under different pressures; wherein MSCEV+P represents extracellular vesicles encapsulating catalase through passive encapsulation, MSCEV+0 mPa represents extracellular vesicles encapsulating catalase under 0 mPa pressure; and MSCEV+300 mPa represents extracellular vesicles encapsulating catalase under 300 mPa pressure;

FIG. 4 is a schematic diagram of the particle size of extracellular vesicles encapsulating the required substance using Lactobacillus as the source of extracellular vesicles and obtained through passive encapsulation and under different pressures; wherein LGEV represents extracellular vesicles of Lactobacillus, CEV+P represents extracellular vesicles encapsulating catalase through passive encapsulation, CEV+0 mPa represents extracellular vesicles encapsulating catalase under 0 mPa pressure, and CEV+300 mPa represents extracellular vesicles encapsulating catalase under 300 mPa pressure;

FIG. 5 is a schematic diagram of the particle size of extracellular vesicles encapsulating the required substance using mesenchymal stem cells as the source of extracellular vesicles and obtained through passive encapsulation and under different pressures; wherein MSC represents extracellular vesicles of mesenchymal stem cells, MSCEV+P represents extracellular vesicles encapsulating catalase through passive encapsulation, MSCEV+0 mPa represents extracellular vesicles encapsulating catalase under 0 mPa pressure; and MSCEV+300 mPa represents extracellular vesicles encapsulating catalase under 300 mPa pressure;

FIG. 6A to FIG. 6D are schematic diagrams of the particle size of extracellular vesicles encapsulating the required substance using Lactobacillus as the source of extracellular vesicles and obtained through passive encapsulation and under different pressures, analyzed with a nanoparticle tracking analyzer; wherein LGEV represents extracellular vesicles of Lactobacillus, CEV+P represents extracellular vesicles encapsulating catalase through passive encapsulation, CEV+0 mPa represents extracellular vesicles encapsulating catalase under 0 mPa pressure, and CEV+300 mPa represents extracellular vesicles encapsulating catalase under 300 mPa pressure; and

FIG. 7A to FIG. 7D are schematic diagrams of the particle size of extracellular vesicles encapsulating the required substance using mesenchymal stem cells as the source of extracellular vesicles and obtained through passive encapsulation and under different pressures, analyzed with a nanoparticle tracking analyzer; wherein MSC represents extracellular vesicles of mesenchymal stem cells, MSCEV+P represents extracellular vesicles encapsulating catalase through passive encapsulation, MSCEV+0 mPa represents extracellular vesicles encapsulating catalase under 0 mPa pressure; and MSCEV+300 mPa represents extracellular vesicles encapsulating catalase under 300 mPa pressure.

### DETAILED DESCRIPTION OF THE PREFERRED EMBODIMENTS

All numerical values in this disclosure can be understood as modified by “about”. In one embodiment, when referring to a measurable value such as a quantity, unless otherwise stated, the term “about” is meant to encompass ±10% of the specified value, such as the conductivity of saliva. As used herein, when referring to a range, the term “about” means ±10% of the difference in the range covering the specified value, unless otherwise stated.

### Example 1

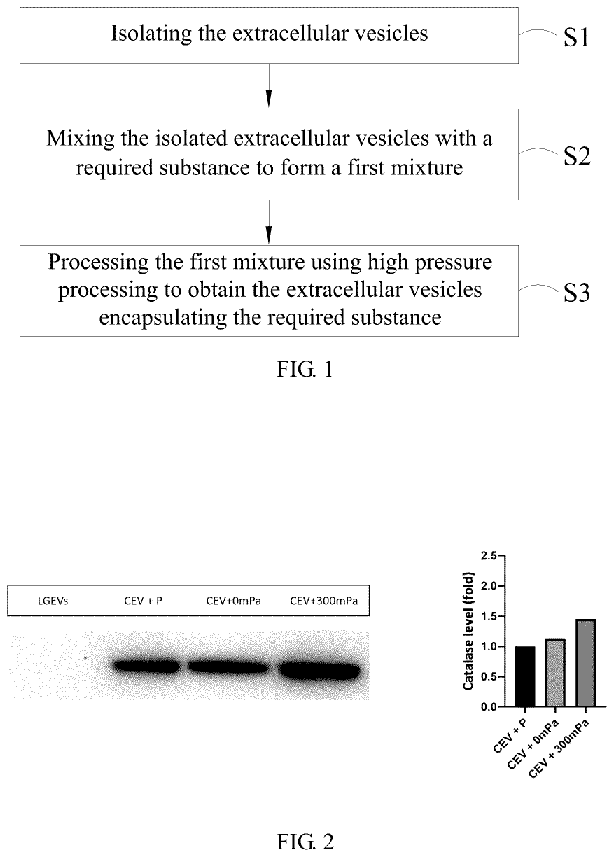

With reference to FIG. 1, the encapsulation method using extracellular vesicles as carriers of the present invention comprises the following steps:

Step S1: isolating the extracellular vesicles.

In step S1, the culture medium containing extracellular vesicles is filtered with a filter with a pore size of 0.22 m, and ultracentrifuged at 4° C. for 4 hours. Discard the supernatant and redissolve the precipitate in phosphate buffer solution (DPBS), ultracentrifuged again at 4° C. for 2 hours, and redissolved in DPBS again to obtain a solution containing extracellular vesicles.

Preferably, considering issues such as safety, mass production, and cost, the source of the extracellular vesicles is Lactobacillus or stem cells (SC); more preferably, the Lactobacillus is Lactobacillus gasseri (L. gasseri, LG), and the stem cells are mesenchymal stem cells (MSC), but is not limited thereto.

In addition, in order to confirm that the novel encapsulation method of the present invention is able to work on different extracellular vesicles, Lactobacillus and mesenchymal stem cells were used as sources of extracellular vesicles for experiment to prove the practicality and efficiency of the of requested novel encapsulation method of the present invention.

Step S2: mixing the isolated extracellular vesicles with a required substance to form a first mixture.

In step S2, the required substance is added to the solution containing the extracellular vesicles obtained in step S1 to form a first mixture, the required substance can be small molecules, peptides, nucleic acids, enzymes, aptamer and/or scaffold, but are not limited thereto. Preferably, the required substance is catalase, which is added at a concentration of 300 ppm and contains 20 g/mL of total protein.

Step S3: processing the first mixture using high pressure processing to obtain the extracellular vesicles encapsulating the required substance.

In step S3, the first mixture is processed with high pressure processing, which is processed for a certain time under a pressure of 0˜600 mPa, to obtain the extracellular vesicles encapsulating the required substance; preferably, the high pressure processing is performed at a pressure of 300 mPa for 10 minutes to obtain the extracellular vesicles encapsulating the required substance.

In addition, in order to examine the effect of pressure on encapsulating efficiency, in step S3, the first mixture was further processed under a pressure of 0 mPa to obtain the extracellular vesicles encapsulating the required substance. Furthermore, in order to examine the effect of traditional passive encapsulation and the use of high pressure processing on encapsulating efficiency, after step S2, the obtained first mixture was left to stand at 37° C. for 1 hour to obtain the extracellular vesicles encapsulating the required substance.

### Results

In Example 1, the extracellular vesicles were obtained from Lactobacillus and mesenchymal stem cells, and the extracellular vesicles encapsulating the required substance were either processed under different pressure or processed through traditional passive encapsulation, and confirm the encapsulating efficiency using Western blotting, electron microscopy and nanoparticle tracking analyzer.

The Western blotting method is to process the extracellular vesicles encapsulating the required substance using protein quantification to unify the protein concentration, then take 5 μg of total protein for electrophoresis to separate the protein, and transfer to a polyvinylidene fluoride (PVDF) film, add primary antibody for labeling, and add secondary antibody for cleaning. Finally, the film is photographed with a multifunctional image analyzer.

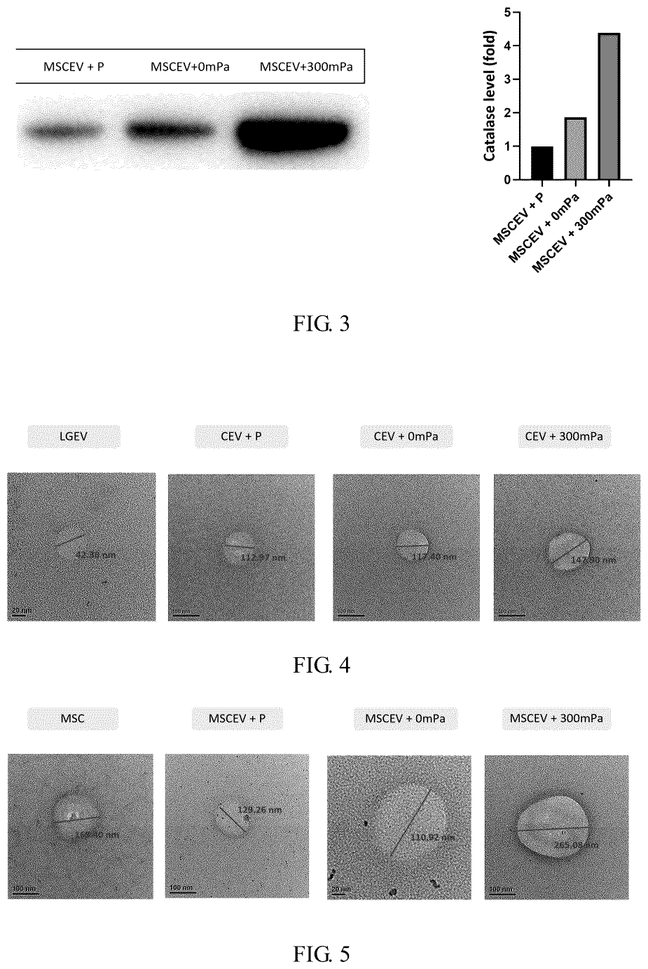

With reference to FIGS. 2 and 3, which in sequence show schematic diagrams of the encapsulating efficiency of extracellular vesicles encapsulating the required substance using Lactobacillus and mesenchymal stem cells as the source of extracellular vesicles and obtained through passive encapsulation and under different pressures. It can be seen from the two figures that in the gel map obtained by the Western ink-dot method, passive encapsulation showed lower encapsulating efficiency than that of high pressure processing (HPP), so the color of the colloid is lighter and smaller. In addition, when setting the catalase level of the passive encapsulation as the benchmark (1 times), the encapsulating efficiency by high pressure processing is better, and whether using Lactobacillus or mesenchymal stem cells as the source of extracellular vesicles, they all show a positive correlation between pressure and encapsulating efficiency. As a result, regardless of the source of extracellular vesicles, those that went through high pressure processing all show better encapsulating efficiency than the passive encapsulation.

Additionally, in order to confirm whether high pressure processing (HPP) will destroy the structure of extracellular vesicles, an electron microscope is used to check whether the structure of extracellular vesicles is intact, wherein the results are shown in FIGS. 4 and 5. The two figures show that either Lactobacillus or mesenchymal stem cells, after high pressure processing, the extracellular vesicles still maintain an intact structure, and the amount of required substance encapsulated have both increased as the pressure increases, resulting in an increase in size of the extracellular vesicles, which further proves that high pressure processing can improve encapsulating efficiency, and since the extracellular vesicles still maintain an intact structure, the yield can also increase, thus allowing for mass-production.

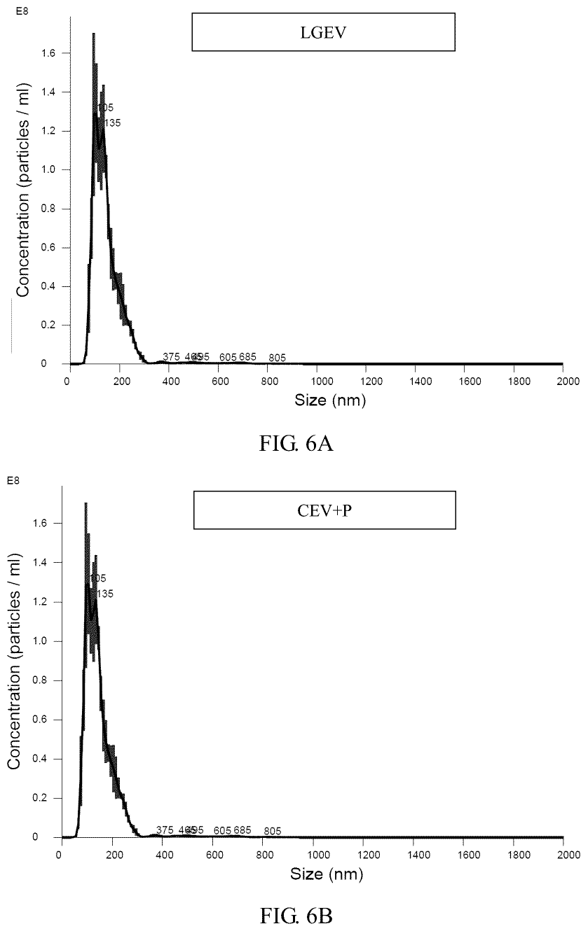

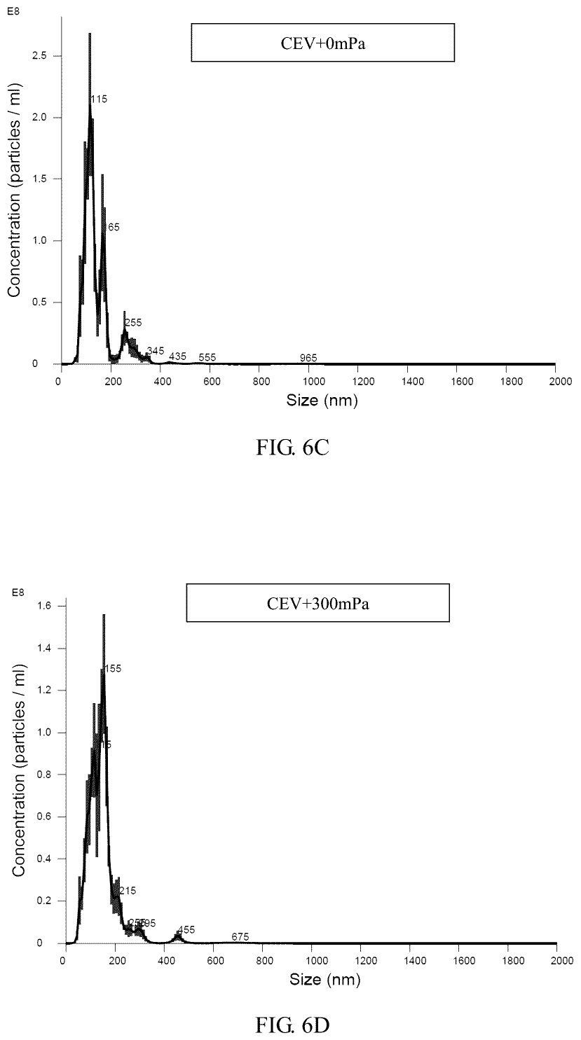

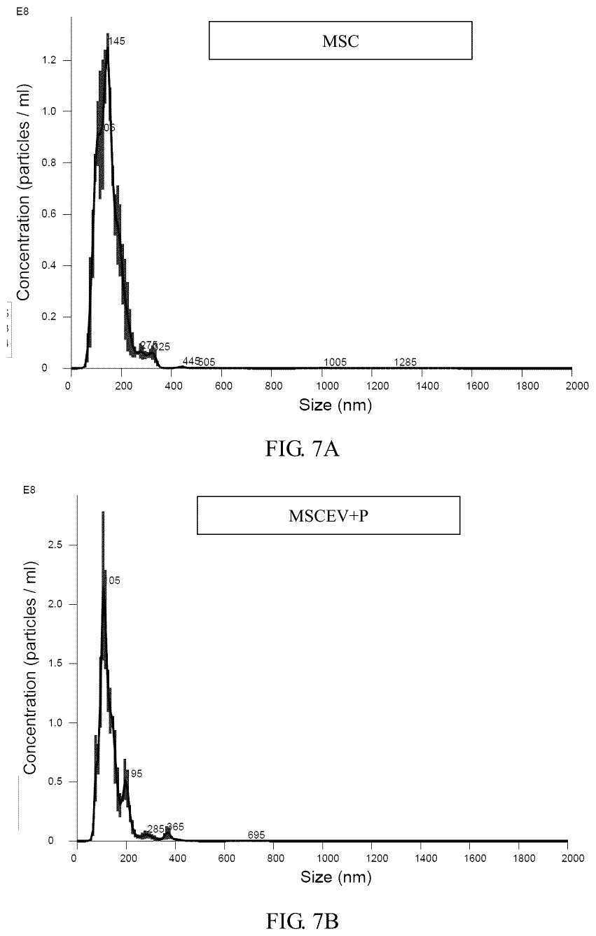

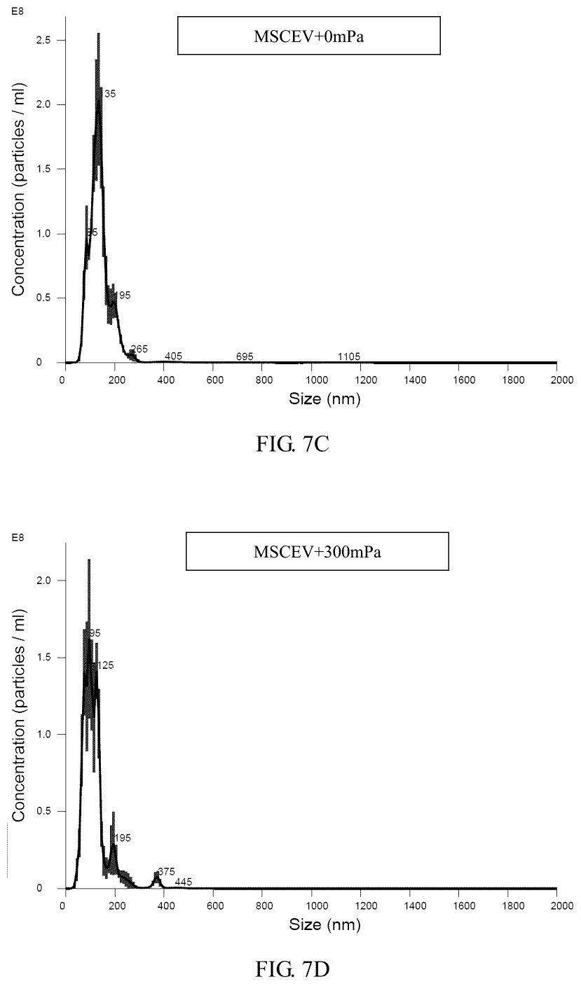

Furthermore, the nanoparticle tracking analyzer is used to conduct particle size analysis on the extracellular vesicles, and the results are as shown in FIGS. 6A to 6D and FIGS. 7A to 7D. As can be seen from the figures, regardless of Lactobacillus or mesenchymal stem cells, the particle size distribution are all within a narrow range. It can further be confirmed that after high pressure processing, its extracellular vesicles have not been damaged but maintain an intact structure. Therefore, introducing high pressure processing into the encapsulation process can indeed improve the encapsulating efficiency and yield without destroying extracellular vesicles, allowing for mass-production. In addition, the particle size of the extracellular vesicles (EVs) obtained from Lactobacillus and mesenchymal stem cells were analyzed in detail by Nanoparticle Tracking Analysis (NTA), which is shown in Table 1 and Table 2 in sequence.

TABLE 1
(extracellular vesicles obtained from Lactobacillus)

| | NTA | | | |
|---|---|---|---|---|
| | | CEV + | CEV + | CEV + |
| EVs | LGEV | P | 0 mPa | 300 mPa |
| Nanoparticle | 147.8 ± 2.0 | 147.8 ± 2.0 | 144.3 ± 1.8 | 147.4 ± 2.8 |
| size (nm) | | | | |

TABLE 2

| | NTA | | | |
|---|---|---|---|---|
| | | MSCEV + | MSCEV + | MSCEV + |
| EVs | MSC | P | 0 mPa | 300 mPa |
| Nanoparticle | 152.1 ± 5.8 | 137.8 ± 4.2 | 138.6 ± 2.1 | 118.0 ± 2.8 |
| size (nm) | | | | |

In summary, introducing high pressure processing into the encapsulation process of extracellular vesicles can solve the problem of low encapsulating efficiency in traditional passive encapsulation process, and can be applied to extracellular vesicles obtained from different sources to achieve same level of efficiency. Therefore, the disclosed method is able to achieve commercialization requirements of encapsulating large amount of drugs with high yields, thus allowing for mass-production.

While the means of specific embodiments in present invention has been described by reference drawings, numerous modifications and variations could be made thereto by those skilled in the art without departing from the scope and spirit of the invention set forth in the claims. The modifications and variations should in a range limited by the specification of the present invention.

What is claimed is:

1. An encapsulation method using extracellular vesicles as carriers, comprising the following steps:
step S1: isolating the extracellular vesicles;
step S2: mixing the isolated extracellular vesicles with a required substance to form a first mixture; and
step S3: processing the first mixture using high pressure processing to obtain the extracellular vesicles encapsulating the required substance.

2. The encapsulation method of claim 1, wherein the extracellular vesicles are obtained from cells or microorganisms.

3. The encapsulation method of claim 1, wherein the required substance is selected from small molecules, peptides, nucleic acids, enzymes, aptamers and/or scaffolds.

4. The encapsulation method of claim 3, wherein the required substance is selected from enzymes.

5. The encapsulation method of claim 4, wherein the enzyme is catalase.

6. The encapsulation method of claim 1, wherein the pressure used in the high pressure processing is 0˜600 mPa.

7. The encapsulation method of claim 6, wherein the pressure used in the high pressure processing is 300 mPa.
